(19) United States
(12) Patent Application Publication
(10) Pub. No.: US 2012/0172900 A1
(43) Pub. Date: Jul. 5, 2012

(54) CELLULOSE SUTURE AND METHOD FOR GRADUAL VESSEL OCCLUSION USING THE SAME

(75) Inventor: Guillaume R. Ragetly, Urbana, IL (US)

(73) Assignee: Guillaume Ragetly, Urbana, IL (US)

(21) Appl. No.: 12/984,221

(22) Filed: Jan. 4, 2011

(51) Int. Cl. A61B 17/00

(52) U.S. Cl. 606/157

(57) ABSTRACT

A method for occluding a vessel includes exposing a vessel and wrapping a cellulose tube suture around the vessel. The cellulose tube includes first and second opposed ends. The first opposed end is fixed to the second opposed end, and the cellulose tube extends around the vessel. The method further includes inflaming the vessel adjacent to the cellulose tube, inflammation of the vessel gradually occluding the vessel.

### TECHNICAL FIELD

Vascular sutures.

### BACKGROUND

Congenital portosystemic shunts are anomalous blood vessels that divert blood from the portal circulation (blood from the intestine and spleen) into the systemic circulation (blood circulating from the heart to the rest of the body). Stated another way, portosystemic shunts divert blood from the liver and thereby allow uncleansed blood to enter they systemic circulation without treatment in the liver. Toxins, such as ammonia found in the portal circulation are able to enter the general circulation of the remainder of the body. Portosystemic shunts are most commonly found in small animals including cats and dogs.

Management of a portosystemic shunt is performed with one or more of surgery, dietary changes and medication each with limitations. In the case of medication and dietary changes symptoms are generally suppressed for a limited period with euthanasia eventually being required. Surgical options that acutely completely shut down blood flow across the portosystemic shunt may cause spikes in blood pressure with the attendant risks of high blood pressure in the intestine.

### BRIEF DESCRIPTION OF THE DRAWINGS

A more complete understanding of the present subject matter may be derived by referring to the detailed description and claims when considered in connection with the following illustrative Figures. In the following Figures, like reference numbers refer to similar elements and steps throughout the Figures.

FIG. 1A is a perspective view of one example of a cellulose tube suture.

FIG. 1B is a perspective view of another example of a cellulose tube suture including feed through orifices at each of the opposed ends of the suture.

FIG. 2 is a perspective view of a deformable clip.

FIG. 3 is a perspective view of one example of a cellulose tube suture kit including a plurality of cellulose tube sutures and a plurality of deformable clips.

FIG. 4 is a perspective view of a cellulose tube suture wrapped around a portion of a vessel.

FIG. 5 is a perspective view of the cellulose tube suture of FIG. 4 wrapped around the vessel with first and second opposed ends fastened together with a deformable clip.

FIG. 6A is a cross sectional view of the vessel immediately after application of the cellulose tube suture.

FIG. 6B is a cross sectional view of the vessel occluded through vessel inflammation some time after application of the cellulose tube suture.

FIG. 7 is a perspective view of one of the opposed ends of the cellulose tube suture positioned within a feed through orifice.

FIG. 8 is a perspective view of the cellulose tube suture of FIG. 7 with the first and second opposed ends fastened together with a deformable clip while the opposed end is positioned within the feed through orifice.

FIG. 9A is a perspective view showing one example of a prior art cellulose strip.

FIG. 9B is a perspective view showing the cellulose strip of FIG. 9A in an intermediate folded configuration.

FIG. 9C is a perspective view showing the cellulose strip of FIG. 9C in a folded configuration.

FIG. 10A is a perspective view of the prior art cellulose strip within a surgical site.

FIG. 10B is a perspective view of the cellulose strip of FIG. 9C fixed with multiple deformable clips around a vessel.

Elements and steps in the Figures are illustrated for simplicity and clarity and have not necessarily been rendered according to any particular sequence. For example, steps that may be performed concurrently or in different order are illustrated in the Figures to help to improve understanding of examples of the present subject matter.

### DESCRIPTION OF THE DRAWINGS

In the following detailed description, reference is made to the accompanying drawings which form a part hereof, and in which is shown by way of illustration specific examples in which the subject matter may be practiced. These examples are described in sufficient detail to enable those skilled in the art to practice the subject matter, and it is to be understood that other examples may be utilized and that structural changes may be made without departing from the scope of the present subject matter. Therefore, the following detailed description is not to be taken in a limiting sense, and the scope of the present subject matter is defined by the appended claims and their equivalents.

The present subject matter may be described in terms of functional block components and various processing steps. Such functional blocks may be realized by any number of techniques, technologies, and methods configured to perform the specified functions and achieve the various results.

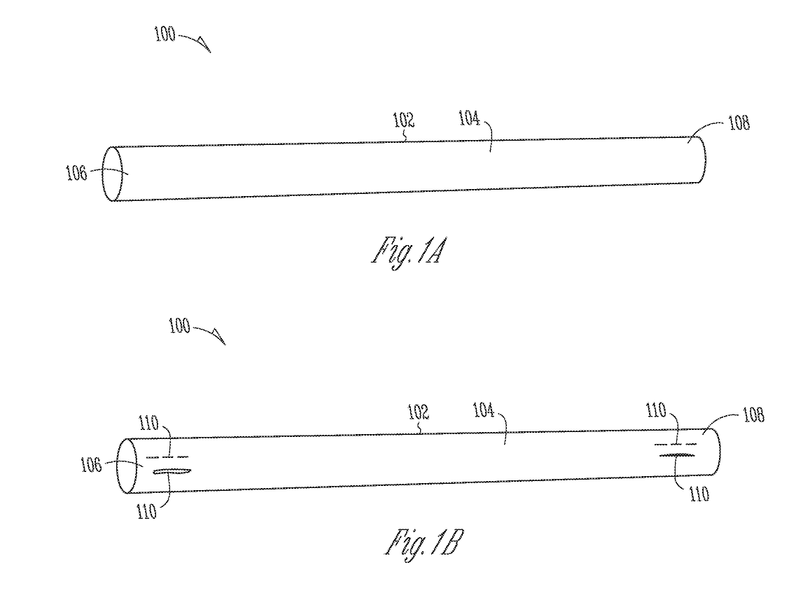

FIGS. 1A and 1B show examples of cellulose tube sutures 100 configured for gradual occlusion of blood vessels including, for instance, portosystemic shunts. Referring first to FIG. 1A, the first cellulose tube suture 100 includes a tube body 102 extending between first and second ends 106, 108. The cellulose tube suture 100 includes a cylindrical perimeter 104 as shown in FIG. 1A. The cylindrical perimeter 104 is substantially continuous in a radial manner thereby forming the cylindrical shape of the cellulose tube suture 100. With the cylindrical perimeter 104 the cellulose tube suture 100 is substantially free of sharp edges along the length of the cellulose tube suture 100. As will be described in further detail below, because the cellulose tube suture 100 includes the cylindrical perimeter 104 a surgeon is able to easily grasp and manipulate the cellulose tube suture 100 to wrap the suture around a vessel and thereafter fasten it together at the first and second opposed ends 106, 108. Additionally, because the cellulose tube suture 100 includes the cylindrical perimeter 104 the tube suture 100 is free of any sharp edges capable of engaging with and causing damage to vasculature and adjacent tissues.

In one example the cellulose tube suture 100 will have a diameter measured across the tube body 102 of between around one and five millimeters. The relatively large diameter of the cellulose tube suture 100 and its cylindrical shape allows the surgeon to easily grasp the cellulose tube suture 100 during an operation within a slick environment, such as in and around the vasculature surrounding the liver of an animal. Additionally, the relatively thick character (e.g. diameter) of the cellulose tube suture 100 allows for the first and second opposed ends 106, 108 to be easily grasped by a clip such as a deformable clip described herein. Further, the cylindrical perimeter 104 provides a robust feature for grasping by the deformable clip. Stated another way it is easier for a deformable clip or for instance a surgeon's hand to grasp a tube having a cylindrical configuration than a folded piece of suture material, such as cellophane.

Referring now to FIG. 1B, another example of the cellulose tube suture 100 is shown. The suture 100 shown in FIG. 1B includes two feed through orifices 110 positioned at each of the first and second ends 106, 108. The feed through orifices 110 are sized and shaped to receive an opposed end 106, 108 therein to facilitate coupling and fastening of the cellulose tube suture 100 around a vessel designated for occlusion. As shown in FIG. 1B, each of the feed through orifices 110 extends through both sides of the cylindrical perimeter 104 of the tube body 102. The surgeon is thereby able to lace one of the first and second ends 106, 108 through the feed through orifice 110 of the opposed end 108, 106. The surgeon may use a tool for clamping the first and second ends 106, 108, such as a hemostat, to lace one of the ends through the feed through orifice 110. After positioning of the first or second end 106, 108 through one of the feed through orifices 110 the surgeon grasps the now protruding end extending out of the feed through orifice 110 and thereafter is able to pull the laced end through the feed through orifice 110 to tighten the cellulose tube suture 100 around a vessel, such as a portosystemic shunt.

Although the feed through orifice 110 shown in FIG. 1B extends through both sides of the tube body 102 of the tube suture 100, in another example the feed through orifice 110 extends through a single side of the cellulose tube suture 100. That is to say, the feed through orifices 110 shown at each of the first and second ends 106, 108 would be consolidated into a single orifice and when one of the first and second ends 106, 108 is fed through the single feed through orifice 110 it is thereafter extended through the tubular end of the tube body 102 to facilitate fastening of the cellulose tube suture 100.

As described herein each of the cellulose tube sutures 100 is constructed with a material including cellulose. For instance the cellulose tube sutures 100 are constructed with but not limited to cellophane, materials incorporating cellophane, materials incorporating cellulose or cellophane in combination with other materials and the like. As described in further detail below, the cellulose present within the cellulose tube suture 100 interacts with the adjacent vessel wall and causes an inflammatory reaction in the vessel wall. Inflammation caused by the cellulose tube suture 100 gradually occludes the vessel and correspondingly gradually closes the vessel over a period of time. Gradual occlusion of the vessel allows the body of the patient to progressively accustom itself to the change in blood flow (elimination of the flow through the vessel and increase of blood flow through other anatomically normal vessels). In the case of a portosystemic shunt the gradual inflammation caused by the cellulose tube suture 100 gradually occludes the portosystemic shunt thereby diverting blood travelling directly from the portal circulation into the systemic circulation. The blood is properly diverted into the liver for interaction with the liver before being delivered into the systemic circulation.

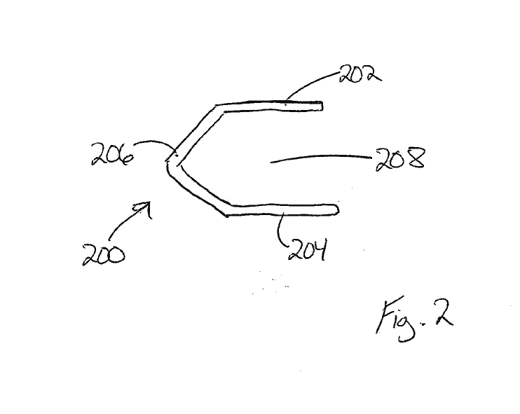

FIG. 2 shows one example of a deformable clip 200 usable with the cellulose tube sutures 100 shown in FIGS. 1A, 1B. The deformable clip 200 includes first and second jaws 202, 204 coupled together at a joint 206. As shown in FIG. 2 a clip mouth 208 is positioned between each of the first and second jaws 202, 204. One example of the deformable clip 200 is constructed with a deformable material such as a metal including but not limited to stainless steel, aluminum and the like. The deformable clip 200 is sized and shaped for easy grasping with an instrument such as a hemostat, specialized pliers or the like. As will be described in further detail below, a deformable clip 200 is operated by positioning the first and second opposed ends 106, 108 of the cellulose tube suture 100 within the clip mouth 208 and thereafter deforming the deformable clip 200 so that the first and second jaws 202, 204 engage with the cellulose tube suture 100 and grasp the cellulose tube suture therebetween. As shown in FIG. 2, the deformable clip 200 is deformed at the joint 206 to facilitate the engagement of the first and second jaws 202, 204 with the cellulose tube suture 100.

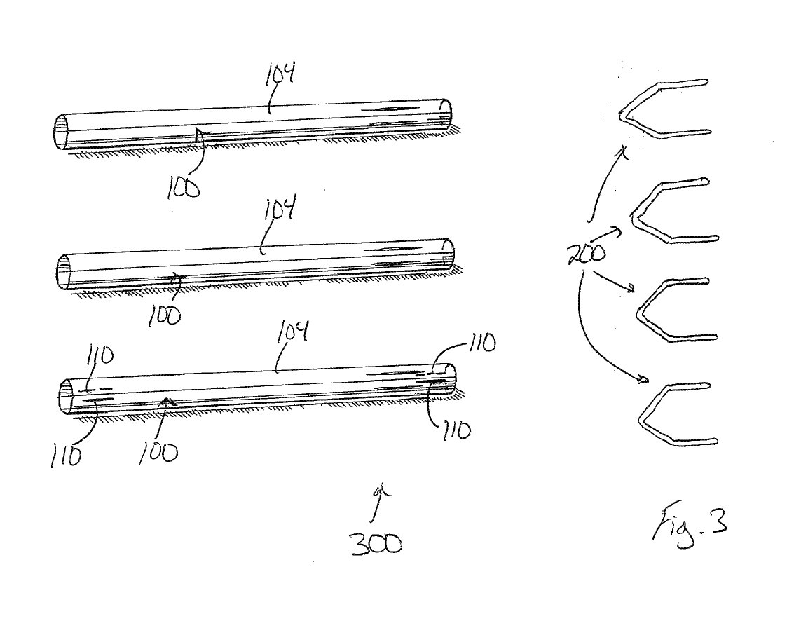

FIG. 3 shows one example of kit 300 for use in the gradual occlusion of vessels. As shown the kit 300 includes one or more cellulose tube sutures 100. As previously described the cellulose tube sutures 100 include a cylindrical perimeter 104 and are configured for wrapping around a desired vessel. After being fastened around the desired vessel one or more of the cellulose tube sutures 100 are configured for gradual occlusion of the vessel through inflammation of the vessel tissue. In the example shown in FIG. 3 a plurality of cellulose tube sutures 100 are included in the kit 300. The first two cellulose tube sutures 100 (from the top down) are cylindrical tube sutures sized and shaped for wrapping around a vessel and thereafter being clipped by the deformable clips 200.

The third cellulose tube suture 100 includes feed through orifices 110 sized and shaped to receive one or more of the opposed ends 106, 108 (see FIG. 1A, 1B) therein. As will be described in further detail below, the cellulose tube suture 100 with the feed through orifices 110 is configured for lacing of one of the ends through a feed through orifice 110 and thereafter fastening the cellulose tube suture together either with the engagement between the opposed end in the feed through orifice of the opposed end 106, 108 or the combination of the feeding through of one end through the feed through orifice 110 along with deformation and engagement of one or more deformable clips 200 at the opposed ends.

In one option, the kit 300 includes a plurality of each of the cellulose tube sutures 100 (sutures without feed through orifices 110 and sutures with feed through orifices 110). In still another option, the kit 300 includes a plurality of cellulose tube sutures 100 having a variety of lengths to facilitate the use of the cellulose tube sutures in a plurality of different anatomies (e.g., for different cats dogs and the like). The surgeon is thereby able to choose the cellulose tube suture best suited to the particular vessel and location of the vessel within the body and use that cellulose tube suture in combination with the included deformable clips 200 to gradually occlude the desired vessel.

In still another option, the cellulose tube sutures 100 in the kit 300 are provided in a variety of materials with some cellulose tube sutures constructed with, for instance cellophane, and other cellulose tube sutures are constructed with a combination of cellophane as well as other materials. The surgeon is thereby able to choose the cellulose tube suture 100 desired for the particular operation according to the material properties of each of the cellulose tube sutures 100 (e.g., mechanical compliance such as elasticity, diameter of the sutures and the like).

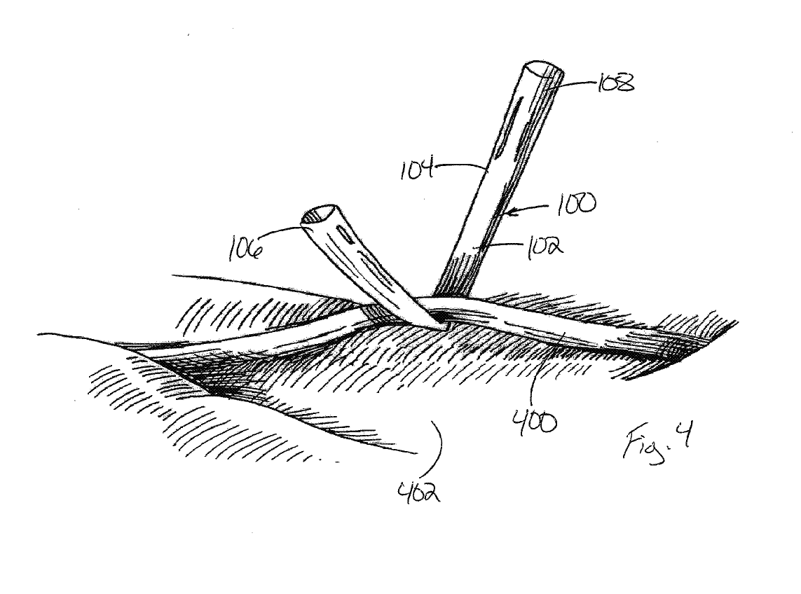

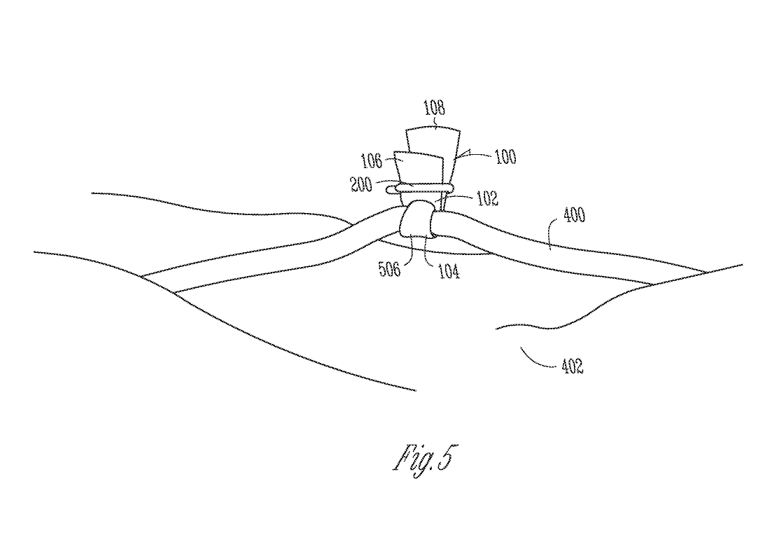

FIGS. 4 and 5 show one method of occluding a vessel, such as vessel 400 within an operating environment 402. In one example, the vessel 400 includes, but is not limited to, a portosystemic shunt. Referring first to FIG. 4 the cellulose tube suture 100 is shown partially wrapped around the vessel 400. The first end 106 and the second end 108 are grasped for instance by hemostats, grasping tools, the surgeon's hands or the like and the first or second end 106, 108 is wrapped underneath the vessel 400 and into the orientation shown in FIG. 4.

As previously described the tube body 102 of the tube suture 100 includes a cylindrical perimeter 104. The cylindrical perimeter 104 ensures the cellulose tube suture 100 has no sharp edges engaged with the vessel 400 in this intermediate configuration prior to fastening of the first and second ends 106, 108. The cellulose tube suture 100 is thereby used within the operating environment 402 with substantially no risk of damage through engagement of a sharp edge with one or more of the vessel 400 or other tissues within the environment 402. As previously described, the tube body 102 has a diameter between around two to three millimeters and is easily grasped with the surgeon's hands or surgical manipulation tool such as hemostats. The cylindrical perimeter 104 provides a robust surface having a tactile tubular shape that is easily grasped whether by the surgeon's hand or a surgical manipulator.

FIG. 5 shows the cellulose tube suture in a closed configuration where the suture 100 is wrapped around the vessel 400 and thereafter engaged at the first and second ends 106, 108 with a deformable clip 200. As shown the cellulose tube suture 102 extends completely around the vessel 400 within the operating environment 402. The cellulose tube suture 100 provides a full loop 506 extending around the cellulose tube suture 400. The loop 506 has a cylindrical perimeter 104 as previously shown in FIGS. 1A, 1B. The cylindrical perimeter 104 is provided by the cellulose tube suture 100 having the tube body 102. The loop 506 with the cylindrical perimeter 104 includes in some examples kinks caused by the wrapping of the cellulose tube suture 100 around the vessel 400. The kinks however only form corners along the cellulose tube suture 100 and are not sharp edges because of the cylindrical perimeter 104 of the tube body 102. Stated another way, the cylindrical perimeter of the loop 506 in some examples is not a perfect cylinder, however it includes a cylindrical perimeter from the perspective that despite having one or more kinks within the loop 506 the cylindrical nature of the loop 506 substantially prevents the presentation of any sharp edges for engagement and damage of the vessel 400 or surrounding tissue within the operating environment 402.

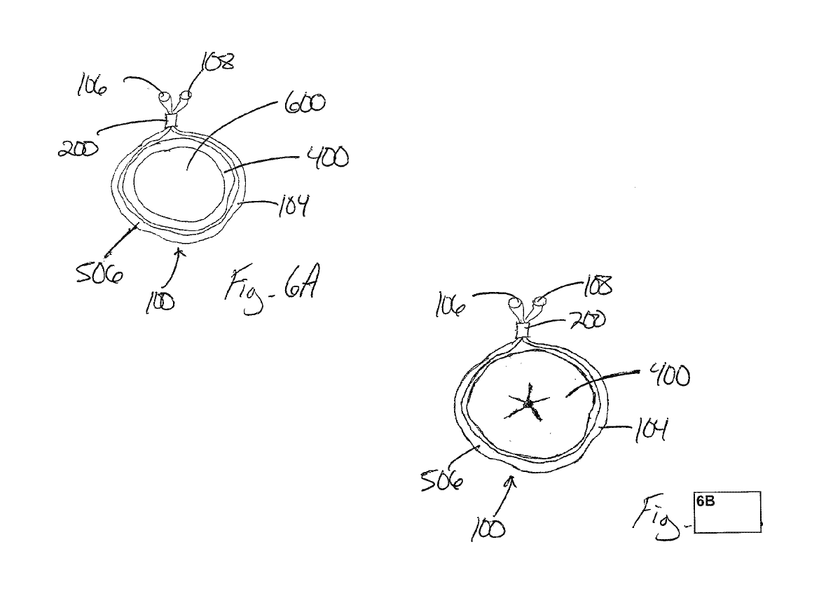

Referring now to FIGS. 6A and 6B, the cellulose tube suture 100 is shown in cross section wrapped around the vessel 400. As shown in FIG. 6A the cellulose tube suture 100 provides a loop 506 extending around the vessel 400 enclosed with the deformable clip 200 engaging the first and second ends 106, 108. As shown in FIG. 6A, cellulose tube suture 100 is not tightly wrapped around the vessel 400 to provide an occluding type engagement. Instead, the cellulose tube suture is wrapped around and loosely engaged with the vessel 400. The vessel passage 600 extends through the vessel 400 and continues to supply blood flow there through.

FIG. 6B shows the cellulose tube suture 100 engaged around the vessel 400 sometime after positioning of the cellulose tube suture 100 in the closed configuration shown in FIG. 5. As previously described, the cellulose tube suture 100 includes a cellulose material configured to initiate an inflammatory reaction in the vessel 400. As shown in FIG. 6B, the wall of the vessel 400 has inflamed and occludes the vessel passage 600 shown in FIG. 6A. Importantly the cellulose tube suture 100 is shown in substantially the same configuration and shape as that provided in FIG. 6A. That is to say, the cellulose tube suture 100 has not been tightened around the vessel 400. Instead, the cellulose material within the cellulose tube suture 100 is relied upon to instigate the inflammatory reaction in the vessel 400 and thereby gradually occlude the vessel passage 600 (see FIG. 6A). The gradual occlusion of the vessel allows the body to adjust to the eventual cessation of blood flow through the vessel 400 thereby substantially preventing complications in the patient including elevated blood pressure and the associated complications that follow with elevated blood pressure.

In one example, where the vessel 400 includes a portosystemic shunt the gradual occlusion of the portosystemic shunt (e.g. vessel 400) allows for the gradual diversion of blood from the vessel passage 600 into the liver thereby facilitating the termination of the portosystemic shunt and directing blood flow from the portal circulation into the liver and then into the systemic circulation. Portosystemic shunts within the patient are thereby closed gradually over a period of time while the blood flow originally passing through the portosystemic shunt is gradually diverted to the liver for treatment and passage into the systemic circulation.

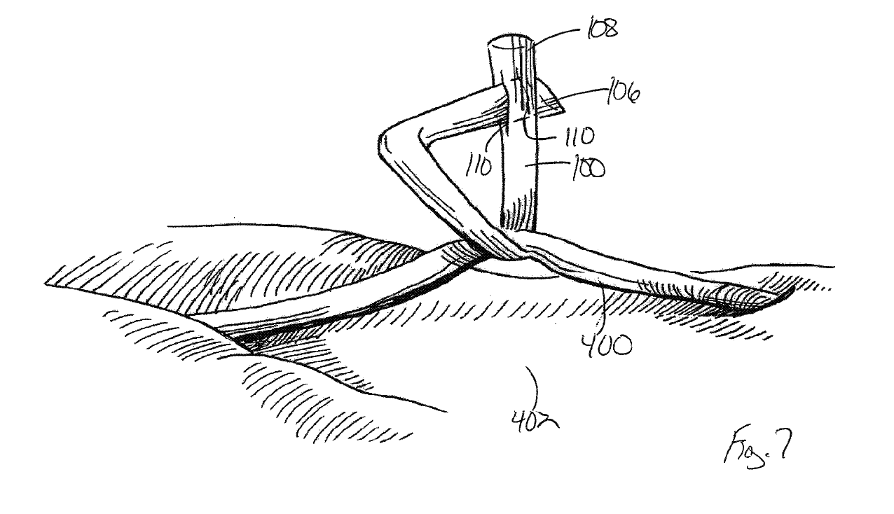

FIG. 7 shows another example of the cellulose tube suture 100. As previously described, the cellulose tube suture 100 includes in one example feed through orifices 110 positioned at one or more of the first and second ends 106, 108. FIG. 7 shows the cellulose tube suture 100 with the feed through orifices 100 in a substantially similar configuration to that shown in FIG. 4. The cellulose tube suture 100 is wrapped around the vessel 400 within the operating environment 402. In this configuration the cellulose tube suture 100 is loosely wrapped around the vessel 400 and the first end 106 is laced through the feed through orifices 110 at the second end 108. As shown in FIG. 7, the feed through orifices 110 are sized and shaped to at least fit the first end 106 of the tube suture 100. In another example the feed through orifices 110 are sized and shaped to receive the first end 106 of the tube suture 100 in a folded configuration to ensure expansion of the cellulose tube suture, after positioning within the feed through orifice 110 (and release by a surgical instrument), engages the first end 106 within the feed through orifice 100 and thereby tightly couples the first end 106 with the second end 108.

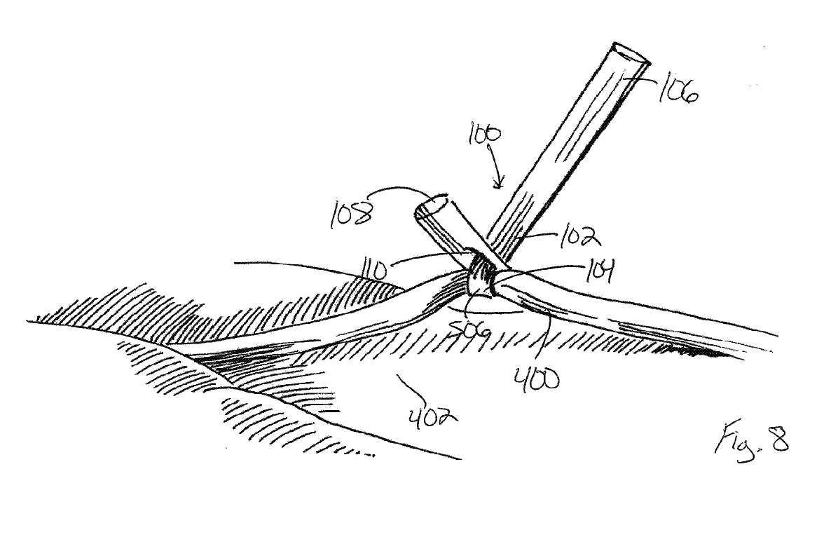

FIG. 8 shows the cellulose tube suture 100 shown in FIG. 7 with the first end 106 pulled through the feed through orifice 110. As shown the cellulose tube suture 100 wraps around the vessel 400 and forms a loop 506 having a cylindrical perimeter 104. As previously described the cylindrical perimeter 104 in one example includes kinks and the like. However any kinks present within the loop 506 are without sharp edges as the cylindrical perimeter 104 of the cellulose tube suture 100 substantially precludes the presentation of sharp edges to the vessel 400 or surrounding tissue in the operating environment 402. As shown in FIG. 8, the first end 106 is pulled through the feed though orifice 110 and presents a trailing piece of the cellulose tube suture 100 extending from the loop 506. In one example, the surgeon severs the extra length of the first end 106 and thereafter fastens the first and second ends 106, 108 together with a deformable clip 200 as shown in FIG. 5. Optionally the deformable clip 200 is engaged with the first and second ends 106, 108 adjacent to the feed through orifice 110. For example the deformable clip 200 extends over the feed through orifice 110 and tightly engages the first and second ends 106, 108 together thereby enhancing the frictional fit provided by the engagement of the first and second ends 106, 108 through the feed through orifice 110. In still another example, multiple deformable clips 200 are applied to the cellulose tube suture 100 to tightly fix the first and second ends 106, 108 together (in either the feed though orifice embodiment or the embodiment without the feed through orifice). In yet another example, a deformable clip 200 is positioned remotely from the feed through orifice 110.

In still another example, the surgeon relies on the frictional engagement of the first and second ends 106, 108 through the feed through orifice 110. As previously described, the first end 106 is passed through the feed through orifice 110 and engages in a frictional fit with the second opposed end 108 by way of expansion of the first end 106 within the feed through orifice 110. Optionally, the cellulose material or cellulose based material of the cellulose tube suture 100 provides a sufficient frictional engagement between the first and second end 106, 108 after passage of one of the ends through the feed through orifice 110. The tube body 102 of the cellulose tube suture 100 is sized and shaped to provide a frictional engagement with the opposed end of the cellulose tube suture having the feed through orifice 110. Stated another way the cylindrical shape of the cellulose tube suture 100 expands within the feed through orifice 110 to provide a tight frictional engagement therein. The cellulose tube suture 100 is thereby firmly held at the first and second ends 106, 108 to form the loop 506 and the suture reliably holds itself around the vessel 400 with or without deformable clips 200.

In much the same manner then, as shown in FIGS. 6A and 6B, after the coupling of the first and second ends 106, 108 through the feed through orifice 110 the cellulose tube suture 100 forms a loop 506 having a substantially cylindrical perimeter 104. Referring first to FIG. 6A, with the loop 506 formed by the cellulose tube suture 100 the vessel 400 is left in a substantially open configuration where the cellulose tube suture extends around and is loosely engaged with the vessel 400. Referring now to FIG. 6B, where the cellulose tube suture 100 including the feed through orifices 110 is left engaged with the vessel 400 over time the vessel 400 inflames according to an interaction with the cellulose and gradually occludes the vessel passage 600 of the vessel 400.

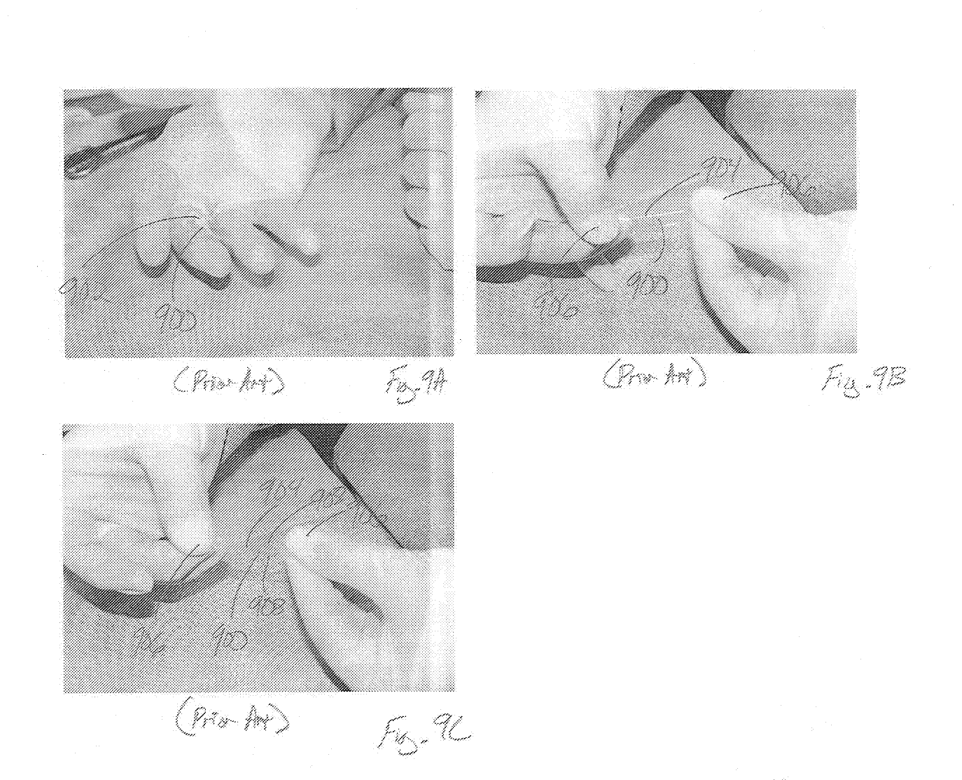

FIGS. 9A-C show one example of a cellophane strip configured for use with a vessel, such as a vessel with an animal body. The cellophane strip 900 is shown with a flat band shape 902 in FIG. 9A. Referring now to FIG. 9B, the user grasps each of the opposed ends of the cellophane strip 900 and folds each of the sides of the band into the configuration shown (an intermediate configuration before full folding of the cellophane strip 900 into a double folded configuration). As shown the surgeon is required to hold each of the opposed ends of the cellophane strip 900 between thumbs and forefingers to maintain the cellophane strip 900 in the shape 904 shown. Because of the natural bias of the material to remain in the band shape shown in FIG. 9A the surgeon must hold the cellophane strip 900 to substantially prevent the unfolding of the cellophane strip into the original configuration.

FIG. 9C shows the cellophane strip 900 in a fully folded configuration 904 where the cellophane strip is folded twice to form the band for positioning around a vessel and occlusion thereof. As shown the band includes sharp edges 908 formed along each of the folds and held in place by the thumb and forefingers of the surgeon 906. Maintaining the cellophane strip 900 in the configuration shown in FIG. 9C is difficult and requires one or more of continuous pressure applied from the thumb and forefinger 906 of the surgeon or the application of pressure through surgical instruments such as hemostats. Additionally, the cellophane configuration 900 includes sharp edges 908 capable of engaging with tissue and vessel structure with the risk of causing damage to the same.

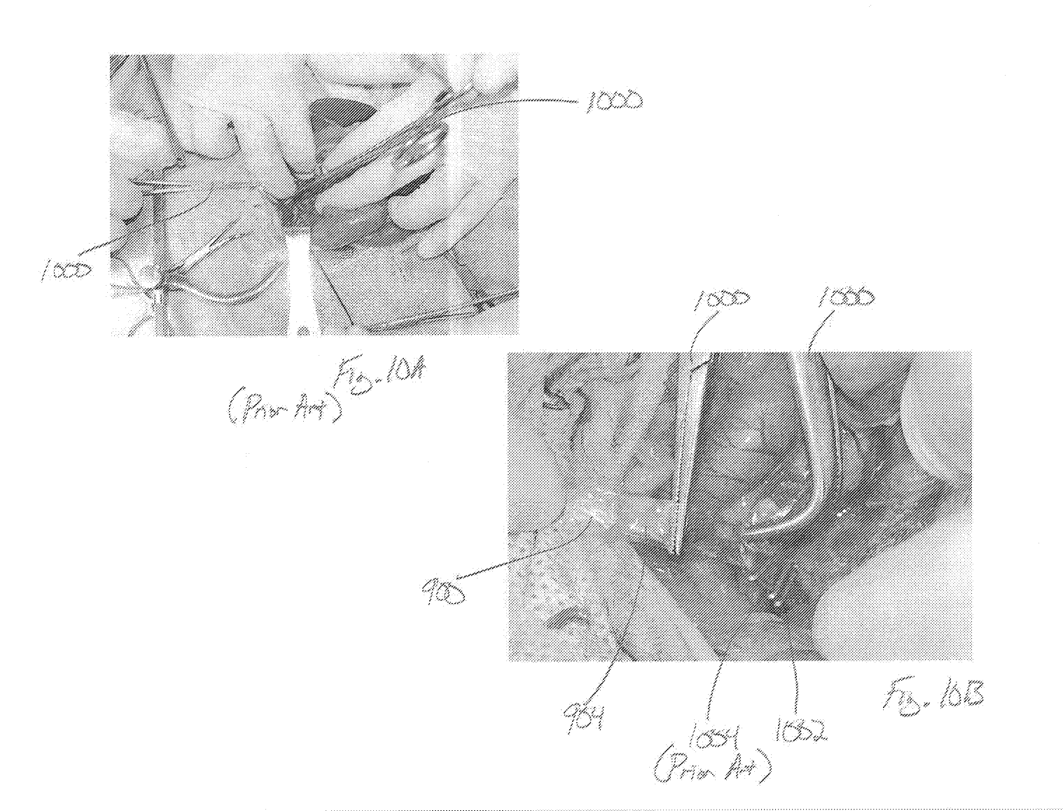

Referring now to FIGS. 10A and 10B, the cellophane strip 900 in the folded configuration 904 is shown alternately positioned around a portion of a vessel 1004 as in FIG. 10A (the vessel is concealed by the surrounding tissues) and fixed around the vessel 1004 in FIG. 10B. As shown, the cellophane strip 900 is held in the folded configuration 904 throughout application and attachment of the opposed ends through one or more of surgical instruments, such as hemostats, or grasping by the surgeon's hands. During application of the prior art cellophane strip it is necessary for the surgeon to alternate between use of the surgical instruments or hands to lace the cellophane strip 900 underneath the vessel and thereafter re-grasp the cellophane strip with one or more of the hands or surgical instruments while clips and the like are applied to the cellophane strip to hold the cellophane strip 900 in place.

As shown in FIG. 10B, the cellophane strip is coupled around the vessel and held with a plurality of deformable clips 1002. The planar nature of the cellophane strip 900 and the folded configuration 904 require the use of multiple clips to provide frictional force to engage the opposed ends of the cellophane strip and hold it around the vessel. Further, even with the use of multiple clips frictional engagement of the opposed ends is not assured. Stated another way, because the cellophane strip 900 does not include inherent thickness and a robust shape the deformable clips are less able to grasp the cellophane strip 900 and hold it in the final configuration shown in FIG. 10B.

### CONCLUSION

The cellulose tube suture described herein provides a robust suture having a cylindrical perimeter. The cellulose tube suture has a thickness (e.g., a diameter) because of the cylindrical configuration that facilitates grasping by a surgeon and engagement with deformable clips to ensure the formation and maintenance of a loop of cellulose around a vessel. Providing a full cellulose loop ensures that inflammation of the entire perimeter of the vessel takes place to gradually occlude the vessel. Additionally, the cylindrical perimeter of the cellulose tube suture provides a tactile sensation to the surgeon while grasping, manipulating and positioning the suture around vessels. Further, the cylindrical perimeter provides tactile sensation to facilitate to grasping of the tube suture for coupling of deformable clips to hold the suture in the loop around a vessel.

Further, the cylindrical perimeter of the cellulose tube suture ensures the suture provides a continuous cylindrical perimeter around a vessel when fixed in a loop configuration. Sharp edges and the like are thereby avoided and the vessel and surgical site are protected from such edges.

Additionally, where the cellulose tube suture includes one or more feed through orifices an opposed end of the cellulose tube suture is easily fed through the orifice to form the loop of the cellulose tube suture around a vessel. In one example, deformable clips are not needed with engagement through the orifice. In another example, a deformable clip is coupled around the opposed ends near to (or remote from) the feed through orifice to provide a reliable coupling between the opposed ends and maintain the loop after positioning around a vessel.

In the foregoing description, the subject matter has been described with reference to specific exemplary examples. However, it will be appreciated that various modifications and changes may be made without departing from the scope of the present subject matter as set forth herein. The description and figures are to be regarded in an illustrative manner, rather than a restrictive one and all such modifications are intended to be included within the scope of the present subject matter. Accordingly, the scope of the subject matter should be determined by the generic examples described herein and their legal equivalents rather than by merely the specific examples described above. For example, the steps recited in any method or process example may be executed in any order and are not limited to the explicit order presented in the specific examples. Additionally, the components and/or elements recited in any apparatus example may be assembled or otherwise operationally configured in a variety of permutations to produce substantially the same result as the present subject matter and are accordingly not limited to the specific configuration recited in the specific examples.

Benefits, other advantages and solutions to problems have been described above with regard to particular examples; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present subject matter, in addition to those not specifically recited, may be varied or otherwise particularly adapted to specific environments, manufacturing specifications, design parameters or other operating requirements without departing from the general principles of the same.

The present subject matter has been described above with reference to examples. However, changes and modifications may be made to the examples without departing from the scope of the present subject matter. These and other changes or modifications are intended to be included within the scope of the present subject matter, as expressed in the following claims.

It is to be understood that the above description is intended to be illustrative, and not restrictive. Many other examples will be apparent to those of skill in the art upon reading and understanding the above description. It should be noted that examples discussed in different portions of the description or referred to in different drawings can be combined to form additional examples of the present application. The scope of the subject matter should, therefore, be determined with reference to the appended claims, along with the full scope of equivalents to which such claims are entitled.

What is claimed is:

1. A method for occluding a vessel comprising:
exposing a vessel;
wrapping a cellulose tube suture around the vessel, wherein the cellulose tube includes first and second opposed ends;
fixing the first opposed end to the second opposed end, and the cellulose tube extends around the vessel; and
inflaming the vessel adjacent to the cellulose tube, inflammation of the vessel gradually occluding the vessel.

2. The method of claim 1, wherein wrapping the cellulose tube suture includes wrapping a cellulose tube suture having a continuous cylindrical perimeter.

3. The method of claim 1, wherein wrapping the cellulose tube suture includes wrapping a cellulose tube having a diameter of around 1 mm to 5 mm.

4. The method of claim 1, wherein fixing the first opposed end to the second opposed end includes clipping the first and second opposed ends with only a single clip.

5. The method of claim 1, wherein fixing the first opposed end to the second opposed end includes feeding a first opposed end through a feed through orifice within the second opposed end.

6. The method of claim 5, wherein fixing the first opposed end to the second opposed end includes clipping the first and second opposed ends at a position of the orifice in the second opposed end.

7. The method of claim 1, wherein exposing the vessel includes exposing a portosystemic shunt blood vessel that bypasses a liver.

8. The method of claim 7, wherein inflaming the vessel adjacent to the cellulose tube includes inflaming the portosystemic shunt blood vessel and occluding the portosystemic shunt blood vessel.

9. The method of claim 8 comprising diverting blood flow from the portosystemic shunt blood vessel to the liver.

10. A kit for occluding a vessel comprising:
one or more cellulose tube sutures, each of the cellulose tube sutures includes:
a continuous cylindrical perimeter, and
first and second opposed ends;
one or more deformable clips, the one or more deformable clips are configured for clipping together first and second opposed ends of the cellulose tube sutures.

11. The kit of claim 10, wherein the one or more cellulose tube sutures include cellophane.

12. The kit of claim 10, wherein the one or more cellulose tube sutures include substantially cylindrical perimeters without sharp edges.

13. The kit of claim 10, wherein the one or more of the first or second opposed ends includes a feed through orifice sized and shaped to receive the other of the first or second opposed ends.

14. The kit of claim 10, wherein the one or more cellulose tube sutures are configured for positioning in open and closed configurations:
in the closed configuration the first and second opposed ends are coupled together, and the cellulose tube suture forms a loop with a cylindrical perimeter, and
in the open configuration the first and second opposed ends are separated, and the cellulose tube suture is configured for wrapping around a vessel.

15. The kit of claim 14, wherein the one or more of the first or second opposed ends includes a feed through orifice sized and shaped to receive the other of the first or second opposed ends, and in the closed configuration one of the first or second opposed ends is positioned within the feed through orifice.

16. The kit of claim 15, wherein in the closed configuration one or more of the clips fasten the first and second opposed ends near the feed through orifice.

17. The kit of claim 14, wherein in the closed configuration one or more of the clips fasten the first and second opposed ends.

18. The kit of claim 10, wherein the continuous cylindrical perimeter has a diameter of around 2 mm to 3 mm.
